(19) United States
(12) Patent Application Publication
(10) Pub. No.: US 2026/0252130 A1
(43) Pub. Date: Aug. 27, 2026

(54) TEMPERATURE CONTROL DEVICE AND TEMPERATURE CONTROL METHOD

(71) Applicant: SONY THERMO TECHNOLOGY INC., Tokyo (JP)

(72) Inventors: Yoichi ITO, Tokyo (JP); Kenji ITOH, Kanagawa (JP)

(73) Assignee: SONY THERMO TECHNOLOGY INC., Tokyo (JP)

(21) Appl. No.: 18/857,459

(22) PCT Filed: Apr. 4, 2023
(86) PCT No.: PCT/JP2023/013887

§ 371 Date: Oct. 17, 2024

(30) Foreign Application Priority Data
Apr. 20, 2022 (JP) 2022-069271

(51) Int. Cl. G05D 23/19; A41D 13/005; A41D 27/28; A41D 31/14; G01K 3/14

(52) U.S. Cl. CPC G05D 23/1931; A41D 27/28; A41D 31/14; G01K 3/14; A41D 13/0051; A41D 13/0053; A41D 13/0058; A41D 2300/20; A41D 2400/12

(57) ABSTRACT

The present technology relates to a wearable-type temperature control device and a temperature control method which can improve the comfort of a user. This temperature control device includes: an environment sensor which measures an environment temperature which is the temperature of the surrounding environment, a temperature changer used for cooling at least the body surface of the user, and a temperature control unit which controls, based on the environment temperature, an element temperature which is the temperature of the temperature changer. The present technology can be used, for example, in a wearable-type temperature control device.

### TECHNICAL FIELD

The present technology relates to a wearable-type temperature control device and a temperature control method and, more particularly, to a temperature control device and a temperature control method configured to improve the comfort of a user.

### BACKGROUND ART

Conventionally, there has been proposed a wearable-type temperature control device that is stored in a pocket provided in an underwear and cools or warms the vicinity of the cervical spine of a user (see, for example, Patent Literature 1).

### CITATION LIST

### Patent Literature

Patent Literature 1: International Publication No. WO 2020/066564

### SUMMARY OF INVENTION

### Technical Problem

In such a wearable-type temperature control device, it is desired to improve the comfort of a user.

The present technology has been devised in view of such a situation and makes it possible to improve the comfort of a user in a wearable-type temperature control device.

### Solution to Problem

A temperature control device according to an aspect of the present technology includes: an environment sensor that measures an environment temperature that is temperature of a surrounding environment; a temperature changer used for cooling at least a body surface of a user; and a temperature control unit that controls, based on the environment temperature, an element temperature that is temperature of the temperature changer.

A temperature control method according to an aspect of the present technology includes a temperature control device: measuring an environment temperature that is temperature of a surrounding environment; and controlling, based on the environment temperature, an element temperature that is temperature of a temperature changer used for cooling at least a body surface of a user.

In an aspect of the present technology, an environment temperature that is temperature of a surrounding environment is measured and an element temperature that is temperature of a temperature changer used for cooling at least a body surface of a user is controlled based on the environment temperature.

### BRIEF DESCRIPTION OF DRAWINGS

FIG. 1 is an exterior view and a sectional view illustrating an embodiment of a temperature control device to which the present technology is applied.

FIG. 2 is a perspective view schematically illustrating an example of a configuration on the front surface side of the inside of a housing of the temperature control device illustrated in FIG. 1.

FIG. 3 is a diagram schematically illustrating an example of a configuration on the rear surface side of the temperature control device illustrated in FIG. 1.

FIG. 4 is a diagram schematically illustrating a configuration of a state in which a fan and the like of the temperature control device illustrated in FIG. 1 are removed.

FIG. 5 is an enlarged view of a part of the temperature control device illustrated in FIG. 4.

FIG. 6 is a block diagram illustrating a configuration example of functions of the temperature control device illustrated in FIG. 1 and the like.

FIG. 7 is a schematic diagram of the exterior of an underwear capable of storing the temperature control device illustrated in FIG. 1 and the like.

FIG. 8 is a state transition diagram of the temperature control device.

FIG. 9 is a diagram for explaining a method of setting an auto start/stop function of a temperature control device.

FIG. 10 is a flowchart for explaining auto start control processing.

FIG. 11 is a diagram for explaining estimation processing for a state of the temperature control device.

FIG. 12 is a flowchart for explaining auto stop control processing.

FIG. 13 is a state transition diagram of the temperature control device in a SMART COOL mode.

FIG. 14 is a diagram illustrating an example of a setting screen for the SMART COOL mode.

FIG. 15 is a diagram illustrating an example of an indicator representing the comfort in clothing.

FIG. 16 is a flowchart for explaining temperature control processing at the time when a SMART COOL mode is set.

FIG. 17 is a diagram illustrating an example of time-series transitions of a heat absorbing surface temperature and a fin temperature at the time when the SMART COOL mode is set.

### DESCRIPTION OF EMBODIMENT

An embodiment of the present technology is explained in detail below with reference to the drawings. Note that the explanation is made in the following order.
1. Embodiment
2. Modifications
3. Others

### 1. Embodiment

### <Configuration Example of the Exterior of a Temperature Control Device 1>

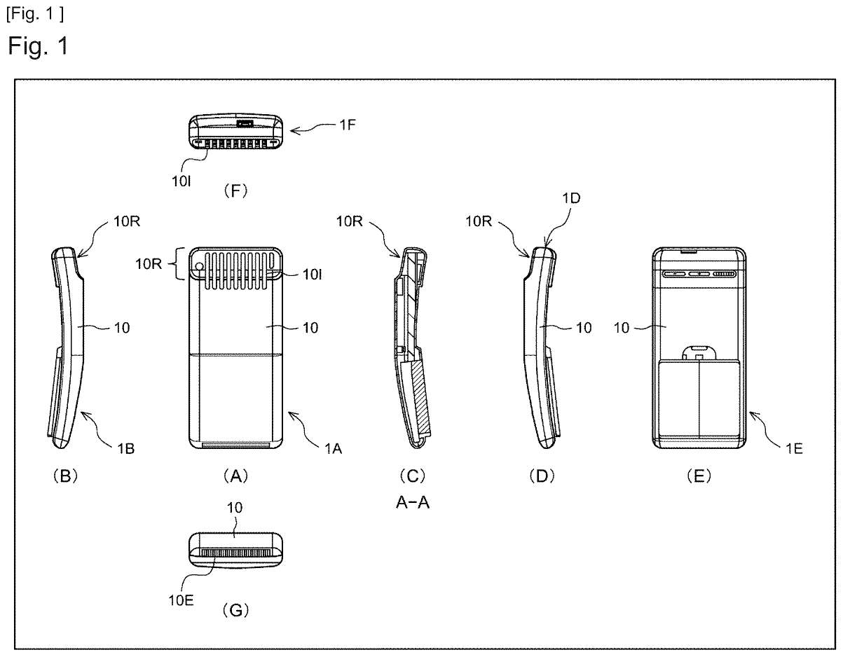

FIG. 1 to FIG. 5 illustrate a configuration example of the exterior of a temperature control device 1 according to an embodiment of the present technology.

A of FIG. 1 illustrates a configuration example of a front surface 1A of the temperature control device 1, B of FIG. 1 illustrates a configuration example of a left side surface 1B of the temperature control device 1, D of FIG. 1 illustrates a configuration example of a right side surface 1D of the temperature control device 1, E of FIG. 1 illustrates a configuration example of a rear surface 1E of the temperature control device 1, F of FIG. 1 illustrates a configuration example of a plane 1F of the temperature control device 1, and G of FIG. 1 illustrates a configuration example of a bottom surface 1G of the temperature control device 1. C of FIG. 1 illustrates a configuration example of a cross section of the right side surface 1D.

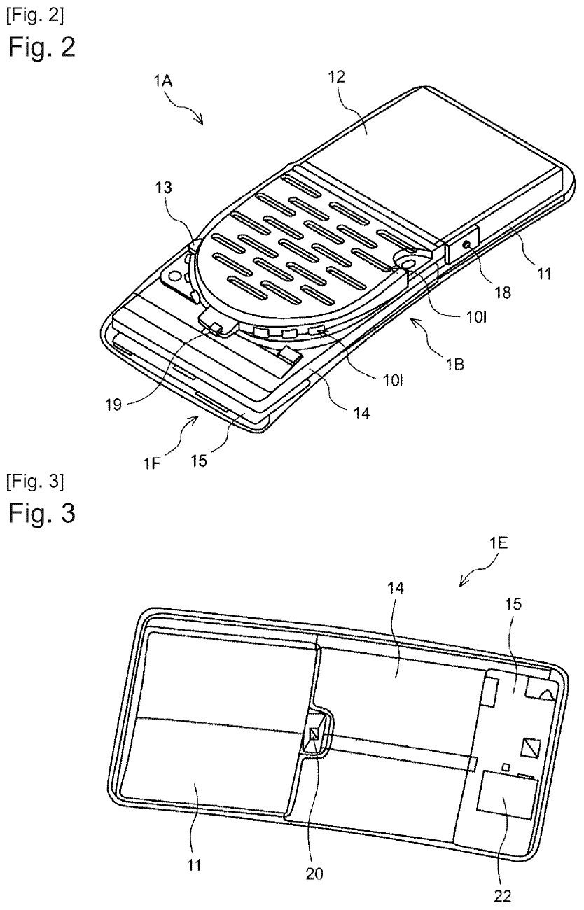

FIG. 2 and FIG. 3 illustrate a configuration example of the temperature control device 1 in a state in which a housing 10 is removed. FIG. 2 is a perspective view illustrating a configuration example of the temperature control device 1 on the front surface 1A side. FIG. 3 illustrates a configuration example of the temperature control device 1 on the rear surface 1E side. Note that the exterior of the temperature control device 1 illustrated in FIG. 1 and the exterior of the temperature control device 1 illustrated in FIG. 2 and FIG. 3 are slightly different. However, the temperature control device 1 may have either one of the exteriors and may have an exterior other than these exteriors.

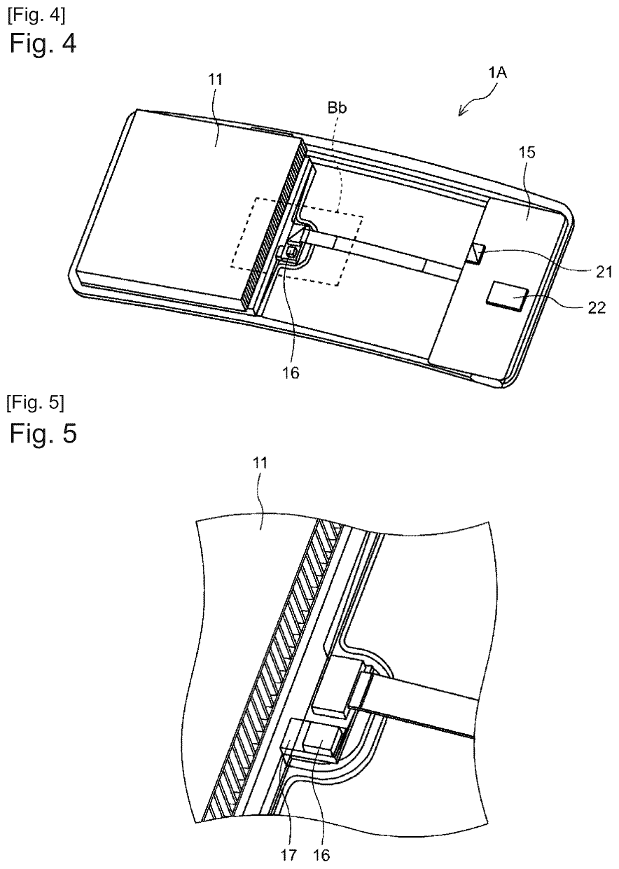

FIG. 4 illustrates a configuration example of the temperature control device 1 on the front surface 1A side in a state in which a fan 13 is removed. FIG. 5 enlarges and illustrates a part (a portion Bb) of FIG. 4.

The temperature control device 1 has, for example, a size equivalent to a business card case and can be stored in a pocket or the like of clothing. That is, the temperature control device 1 is a wearable-type temperature control device that a user can wear and carry.

The temperature control device 1 includes a housing 10 that covers a Peltier element 11. The housing 10 is made of, for example, silicon or a resin material. Examples of the resin material include ABS resin (Acrylonitrile Butadiene Styrene copolymer synthetic resin) and PC (Poly Carbonate) resin.

The front surface 1A and the rear surface 1E of the temperature control device 1 have a rectangular shape. The left side surface 1B and the right side surface 1D of the temperature control device 1 corresponding to long sides of the rectangular shape are gently curved. The shape of the curve conforms to a curve of the shape of a human body that is the user, in particular, the shape near the back and near the neck. Accordingly, the temperature control device 1 can be appropriately applied to a wearing part of the user and can effectively cool or warm the wearing part of the user.

The temperature control device 1 includes, on the inner side of the housing 10, for example, the Peltier element 11, a fin 12, the fan 13, a battery 14, a circuit board 15, a heat absorbing surface sensor 16, a fin sensor 18, an environment sensor 19, and a body surface sensor 20.

The Peltier element 11 is an element that performs temperature control by feeding an electric current. For example, as illustrated in FIG. 2 and FIG. 3, one main surface side of the Peltier element 11 is disposed on the front surface 1A side of the temperature control device 1 and the other main surface side of the Peltier element 11 is disposed on the rear surface 1E side of the temperature control device 1. For example, when the temperature control device 1 performs a cooling operation, the one main surface side of the Peltier element 11 serves as a heat dissipation side and the other main surface side of the Peltier element 11 serves as a heat absorption side. The Peltier element 11 is protected by, for example, silicon. The Peltier element 11 can adjust temperature between, for example, approximately −20° C. and 200° C. The Peltier element 11 is electrically connected to the circuit board 15. The temperature control is performed according to a signal from the circuit board 15.

As illustrated in FIG. 4 and FIG. 5, the heat absorbing surface sensor 16 is provided near a side surface of the Peltier element 11. The heat absorbing surface sensor 16 and the Peltier element 11 are connected by a heat conduction member 17. The heat absorbing surface sensor 16 measures, for example, the temperature on the heat absorption side of the Peltier element 11 (hereinafter referred to as heat absorbing surface temperature) and supplies a sensor signal corresponding to the measured temperature to the circuit board 15. The heat conduction member 17 is made of, for example, silicon, acrylic, graphite, or copper foil.

As illustrated in FIG. 2, the fin 12 is disposed on the heat dissipation side of the Peltier element 11 and dissipates heat on one side of the Peltier element 11. For example, the front surface 1A side of the Peltier element 11 is covered by the fin 12. The fin 12 is, for example, a rectangular plate-like member and is configured by a heat dissipation fin made of aluminum (Al), copper (Cu) or the like.

For example, as illustrated in FIG. 2, the fin sensor 18 is attached to the left side surface 1B side of the fin 12. The fin sensor 18 measures the temperature of the fin 12 (hereinafter referred to as fin temperature) and supplies a sensor signal corresponding to the measured temperature to the circuit board 15.

As illustrated in FIG. 2, the fan 13 is disposed near the fin 12 and ventilates the periphery of the fin 12. The fan 13 is disposed, for example, in a position adjacent to the Peltier element 11 and the fin 12 in the long side direction in the temperature control device 1 having a rectangular flat shape. The fan 13 is disposed, for example, substantially in parallel to the front surface 1A of the temperature control device 1. The fan 13 is a fan for cooling the fin 12 and desirably has, for example, a thin shape. By using the fan 13 having the thin shape, the temperature control device 1 can be reduced in thickness.

As illustrated in FIG. 1 and FIG. 2, a plurality of intake ports 10I are provided in the housing 10 on the front surface 1A side of the fan 13. Outside air is sucked from the intake ports 10I by the fan 13 and blown to the fin 12. For example, as illustrated in FIG. 1, a recess 10R is provided on one short side of the housing 10 on the front surface 1A side and the intake ports 10I is provided in the recess 10R. For example, the housing 10 in a portion adjacent to the fan 13 (a portion on the side opposite to the fin 12) is disposed further on the rear surface 1E side than the housing 10 on the front surface 1A side of the fan 13. A step is provided between the housing 10 in the portion adjacent to the fan 13 and the housing 10 on the front surface 1A side of the fan 13. The intake ports 10I are desirably provided in the step as well. Accordingly, even if sufficient air intake cannot be performed because the intake ports 10I provided in the housing 10 on the front surface 1A side of the fan 13 adhere to clothing or the like, air intake can be performed from the intake ports 10I in the step.

As illustrated in E of FIG. 1, exhaust ports 10E are provided on the bottom surface 1G of the housing 10. Wind sent from the fan 13 and having cooled the fin 12 is blown out by the exhaust ports 10E.

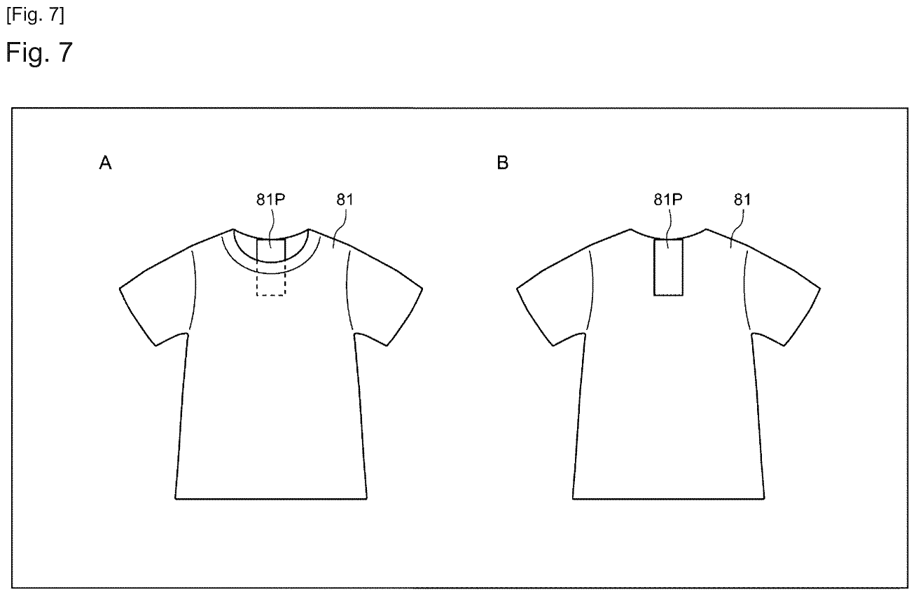

For example, as illustrated in FIG. 2, the environment sensor 19 is disposed near the intake ports 101. A part of the environment sensor 19 is exposed from the housing 10. The part of the environment sensor 19 exposed from the housing 10 is covered by a waterproof sheet (not illustrated). The waterproof sheet allows the air to pass. The environment sensor 19 measures the temperature of an environment (hereinafter referred to as environment temperature) and humidity of the environment (hereinafter referred to as environment humidity) in the periphery where the temperature control device 1 is disposed and supplies a sensor signal corresponding to the measured temperature and the measured humidity to the circuit board 15. When the temperature control device 1 is stored in, for example, a pocket of an underwear (a pocket 81P in FIG. 7 explained below) as explained below, the environment sensor 19 measures the temperature and the humidity in clothing (between the body surface of the user and the clothing).

Note that the environment sensor 19 only has to be capable of measuring at least the environment temperature.

As illustrated in FIG. 2 and FIG. 3, for example, the battery 14 is provided on the rear surface 1E side of the fan 13. The battery 14 is electrically connected to the Peltier element 11, the fan 13, the circuit board 15, and the like and supplies a power supply voltage. The battery 14 has, for example, a rectangular flat shape.

As illustrated in FIG. 3, the body surface sensor 20 is disposed, for example, on the rear surface 1E side of the battery 14. The body surface sensor 20 measures the temperature near the body surface of the user (hereinafter referred to as body surface temperature) and the humidity near the body surface of the user (hereinafter Referred to As Body Surface Humidity) and supplies a sensor signal corresponding to the measured temperature and the measured humidity to the circuit board 15. The body surface sensor 20 may be covered by a waterproof sheet (not illustrated). The body surface sensor 20 is disposed, for example, in a position not overlapping the circuit board 15.

Note that the body surface sensor 20 only has to be capable of measuring at least the body surface temperature of the user.

As illustrated in FIG. 2 and FIG. 3, the circuit board 15 is disposed, for example, further on rear surface 1E side than the battery 14. The circuit board 15 supplies driving signals to the Peltier element 11 and the fan 13 according to the sensor signals and the like supplied from the heat absorbing surface sensor 16, the fin sensor 18, the environment sensor 19, and the body surface sensor 20. The circuit board 15 has, for example, a rectangular shape smaller than the battery 14 and is disposed to overlap the battery 14.

As illustrated in FIG. 3, an acceleration sensor 21 is provided on the circuit board 15. The acceleration sensor 21 measures the acceleration of the temperature control device 1. A state of the user such as a walking state can be predicted by a measurement value of the acceleration sensor 21.

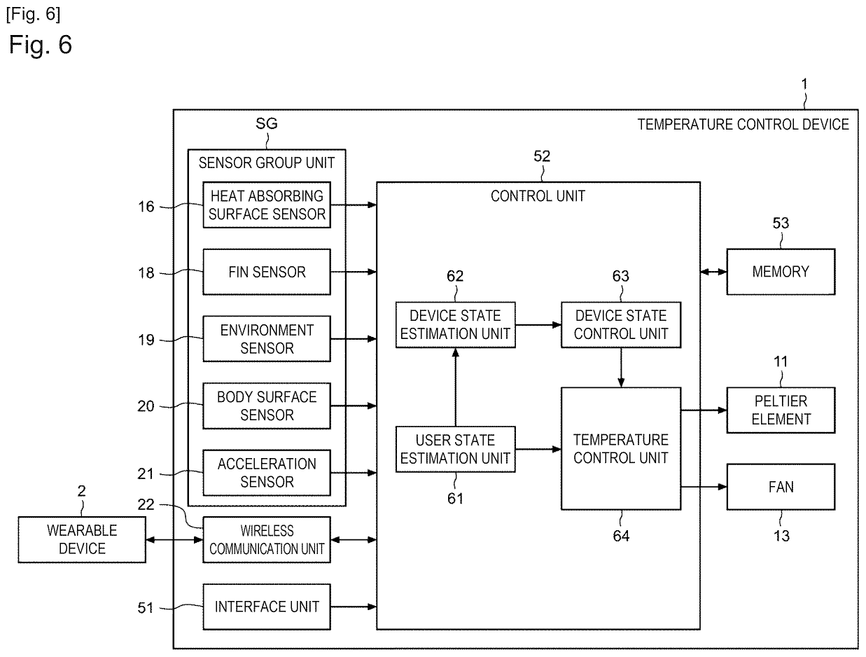

As illustrated in FIG. 3, for example, a wireless communication unit 22 is mounted on the circuit board 15. The wireless communication unit 22 performs wireless communication by, for example, BLE (Bluetooth Low Energy). Since the wireless communication unit 22 is mounted on the circuit board 15, the temperature control device 1 is capable of performing wireless communication with wearable device 2 (FIG. 6) different from the temperature control device 1.

### <Configuration Example of Functions of the Temperature Control Device 1>

FIG. 6 illustrates a configuration example of functions of the temperature control device 1.

Besides the configurations explained above, the temperature control device 1 includes an interface unit 51, a control unit 52, and a memory 53.

The heat absorbing surface sensor 16, the fin sensor 18, the environment sensor 19, the body surface sensor 20, and the acceleration sensor 21 configuring a sensor group SG respectively supply sensor signals to the control unit 52.

The wireless communication unit 22 performs wireless communication with the wearable device 2. The wireless communication unit 22 supplies a communication signal received from the wearable device 2 to the control unit 52 and acquires, from the control unit 52, a communication signal transmitted to the wearable device 2.

The wearable device 2 includes, for example, a smartphone or a smartwatch. For example, the wearable device 2 can be used for various kinds of operation for the temperature control device 1 such as power on/off, temperature setting, and operation mode setting.

For example, the wearable device 2 may receive, from the temperature control device 1, operation data (explained below) concerning an operation of the temperature control device 1 and execute various kinds of processing based on the operation data. For example, the wearable device 2 may display an operation state and the like of the temperature control device 1 and perform temperature control for the temperature control device 1 based on the operation data. For example, the wearable device 2 may learn, based on the operation data, temperature control adjusted to physiological characteristics, lifestyle habits, and the like of individual users.

The interface unit 51 is configured by, for example, buttons. For example, the user can select an operation mode of the temperature control device 1 via the interface unit 51. Operation modes of the temperature control device 1 include, for example, a COOL mode, a WARM mode, and a SMART COOL mode. The COOL mode is a mode in which the temperature control device 1 performs the cooling operation. The WARM mode is a mode in which the temperature control device 1 performs a warming operation. The SMART COOL mode is a mode for automatically adjusting a cooling temperature according to, for example, a surrounding environment of the temperature control device 1 and a state of the user.

When the operation mode is set to the COOL mode, the user can perform temperature setting at predetermined stages (for example, four stages) in a cooling direction with the interface unit 51. When the operation mode is set to the WARM mode, the user can perform temperature setting at predetermined stages (for example, four stages) in a warming direction with the interface unit 51.

The interface unit 51 supplies an operation signal indicating operation content of the user to the control unit 52.

The control unit 52 is implemented by, for example, the circuit board 15. The control unit 52 includes a user state estimation unit 61, a device state estimation unit 62, a device state control unit 63, and a temperature control unit 64.

The user state estimation unit 61 estimates a state of the user based on, for example, the sensor signals from the sensor group SG. For example, the user state estimation unit 61 estimates a behavior, a posture, and the like of the user as the state of the user. The user state estimation unit 61 supplies information indicating an estimation result of the state of the user to the device state estimation unit 62 and the temperature control unit 64.

The device state estimation unit 62 estimates a state of the temperature control device 1 based on, for example, the sensor signals from the sensor group SG and the state of the user estimated by the user state estimation unit 61. For example, the device state estimation unit 62 estimates a posture of the temperature control device 1, presence or absence of wearing by the user, and the like as the state of the temperature control device 1. The device state estimation unit 62 supplies information indicating an estimation result of the state of the temperature control device 1 to the device state control unit 63.

The device state control unit 63 controls the state of the temperature control device 1 based on the sensor signals from the sensor group SG, the communication signal from the wearable device 2, the operation signal from the interface unit 51, the estimation result of the state of the temperature control device 1 by the device state estimation unit 62, and the like. For example, the device state control unit 63 controls a power supply state, an operation mode, an internal state, and the like of the temperature control device 1. The device state control unit 63 supplies information indicating a result of the state control for the temperature control device 1 to the temperature control unit 64. For example, the device state control unit 63 instructs, according to the state of the temperature control device 1 and the like, the temperature control unit 64 to start or stop the cooling operation or the warming operation.

The temperature control unit 64 executes the temperature control for the temperature control device 1 by controlling the Peltier element 11 and the fan 13 based on the sensor signals from the sensor group SG, the communication signal from the wearable device 2, the operation signal from the interface unit 51, the estimation result of the state of the user by the user state estimation unit 61, the result of the state control for the temperature control device 1 by the device state control unit 63, and the like.

The memory 53 is configured by, for example, a NAND flash memory or a NOR flash memory. The memory 53 stores, for example, operation data concerning an operation of the temperature control device 1. The operation data includes, for example, data input from the wearable device 2, the interface unit 51, and the sensor group SG to the control unit 52. The operation data includes, for example, histories of the state of the user estimated by the user state estimation unit 61, the state of the temperature control device 1 estimated by the device state estimation unit 62, the state control for the temperature control device 1 by the device state control unit 63, and the temperature control for the temperature control device 1 by the temperature control unit 64.

For example, the temperature control unit 64 is capable of learning, based on the operation data stored in the memory 53, temperature control adjusted to physiological characteristics, lifestyle habits, and the like of individual users.

### <Example of a Method of Wearing the Temperature Control Device 1>

Subsequently, an example of a method of wearing the temperature control device 1 is explained with reference to FIG. 7.

A of FIG. 7 and B of FIG. 7 schematically illustrate a configuration of an underwear 81 including a pocket 81P for storing the temperature control device 1. A of FIG. 7 illustrates a chest side of the underwear 81 and B of FIG. 7 illustrates a back side of the underwear 81.

The underwear 81 is desirably formed by a material that improves effects of the temperature control device 1. For example, the underwear 81 is made of polyester or the like and has high air permeability and high airtightness. The underwear 81 includes the pocket 81P in a position disposed near the cervical spine when the user wears the underwear 81. That is, the pocket 81P is provided around the neck of the underwear 81. The pocket 81P may be provided on a contact surface side with the skin of the user (the body surface side of the user) (A of FIG. 7) or may be provided on a noncontact surface side with the skin of the user (a side opposite to the body surface of the user) (B of FIG. 7). The depth of the pocket 81P is, for example, equivalent to the size of the long side of the temperature control device 1.

In order to efficiently suck the outside air from the intake ports 101 (FIG. 2) and exhaust from the exhaust ports 10E (FIG. 4), a ventilation hole is desirably provided near the pocket 81P in which the temperature control device 1 is stored. For example, texture is rougher near the pocket 81P than the other portions of the underwear 81. A hole may be opened on the body surface side of the pocket 81P. Since the temperature control device 1 directly touches the skin of the user through the hole, the user easily feels a cooling effect or a warming effect of the temperature control device 1. Thus, the comfort of the user can be improved.

The temperature control device 1 is stored in the pocket 81P such that, for example, the long side direction is vertical and the rear surface 1E side faces the body surface side of the user. The vicinity of the cervical spine of the user is cooled or warmed by the Peltier element 11.

As explained above, users in wide age brackets can easily and casually wear the temperature control device 1. The temperature control device 1 has, for example, the size equivalent to a business card case and can be stored in the pocket 81P of the underwear 81. Therefore, the user can wear the temperature control device 1 without spoiling fashionability.

### <Processing of the Temperature Control Device 1>

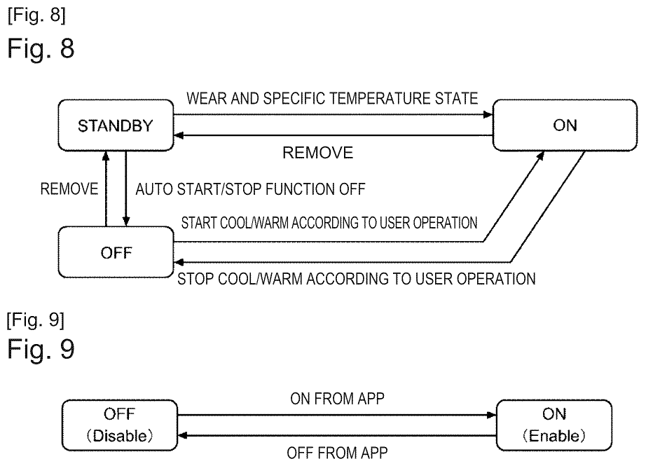

Subsequently, processing of the temperature control device 1 is explained with reference to FIG. 8 to FIG. 17.

### <State Control Processing>

First, state control processing executed by the temperature control device 1 is explained with reference to FIG. 8 to FIG. 12.

FIG. 8 illustrates a state transition diagram of the temperature control device 1.

The temperature control device 1 transitions among three states of OFF, ON, and standby.

The OFF state is a state in which the temperature control device 1 has been turned off and has stopped operating.

The ON state is a state in which the temperature control device 1 has been turned on and is operating. Specifically, the ON state is a state in which the temperature control device 1 is performing cooling or warming or has temporarily stopped cooling or warming.

The standby state is a state in which the temperature control device 1 has been turned off and has stopped operating and is a state in which the temperature control device 1 is capable of automatically transitioning to the ON state without depending on user operation in the case in which a predetermined condition is satisfied. The standby state is a state to which the temperature control device 1 transitions only when an auto start/stop function has been set to on.

In the case of the OFF state, the temperature control device 1 transitions to the ON state when user operation for starting COOL (cooling) or WARM (warming) is performed using the interface unit 51 or the wearable device 2.

In the case of the OFF state, the auto start/stop function has been set to on and, when being removed from the user, the temperature control device 1 transitions to the standby state.

In the case of the ON state, the temperature control device 1 transitions to the OFF state when user operation for stopping COOL or WARM is performed using the interface unit 51 or the wearable device 2.

In the case of the ON state, the auto start/stop function has been set to on and, when being removed from the user, the temperature control device 1 transitions to the standby state.

In the case of the standby state, the temperature control device 1 is worn by the user and transitions to the ON state in a specific temperature state.

Here, when the COOL mode or the SMART COOL mode is set, the specific temperature state is, for example, a case in which the environment temperature is high, a case in which the environment temperature is sharply rising, a case in which the body surface temperature is high, or a case in which the body surface temperature is sharply rising. The case in which the environment temperature is high is, for example, a case in which the environment temperature is equal to or higher than a predetermined threshold. The case in which the environment temperature is sharply rising is, for example, a case in which a rising rate of the environment temperature is equal to or higher than a predetermined threshold. The case in which the body surface temperature is high is, for example, a case in which the body surface temperature is equal to or higher than a predetermined threshold. The case in which the body surface temperature is sharply rising is, for example, a case in which a rising rate of the body surface temperature is equal to or higher than a predetermined threshold.

When the WARM mode is set, the specific temperature state is, for example, a case in which the environment temperature is low, a case in which the environment temperature is sharply falling, a case in which the body surface temperature is low, or a case in which the body surface temperature is sharply falling. The case in which the environment temperature is low is, for example, a case in which the environment temperature is lower than the predetermined threshold. The case in which the environment temperature is sharply falling is, for example, a case in which a falling rate of the environment temperature is equal to or higher than a predetermined threshold. The case in which the body surface temperature is low is, for example, a case in which the body surface temperature is equal to or lower than the predetermined threshold. The case in which the body surface temperature is rapidly falling is, for example, a case in which a falling rate of the body surface temperature is equal to or higher than a predetermined threshold.

In the case of the standby state, the temperature control device 1 transitions to the OFF state when the auto start/stop function is turned off.

FIG. 9 illustrates a method of setting the auto start/stop function of the temperature control device 1. For example, the user can turn on or off the auto start/stop function using predetermined application software (APP) on the wearable device 2.

### <Auto Start Control Processing>

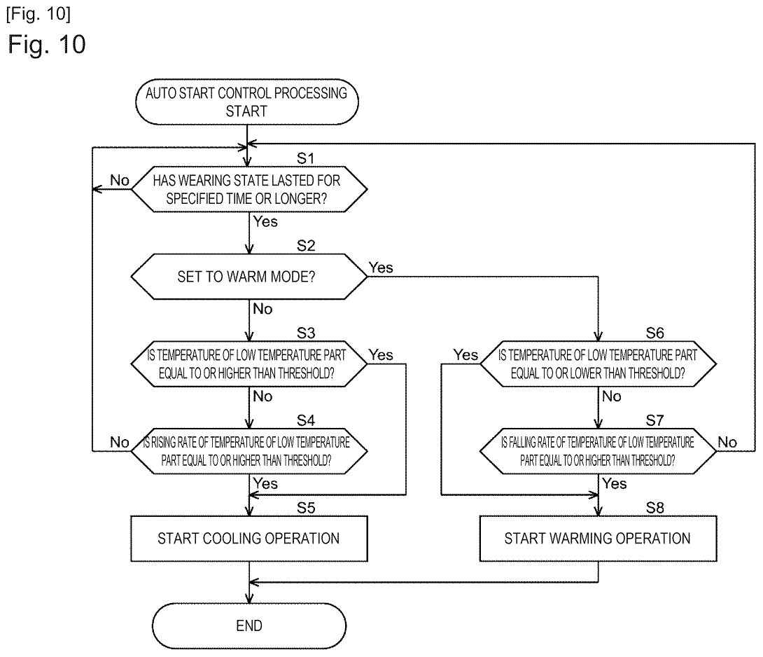

Subsequently, auto start control processing executed by the temperature control device 1 is explained with reference to a flowchart of FIG. 10. The processing is processing executed at the time of transition from the standby state to the ON state in the state transition diagram of FIG. 8.

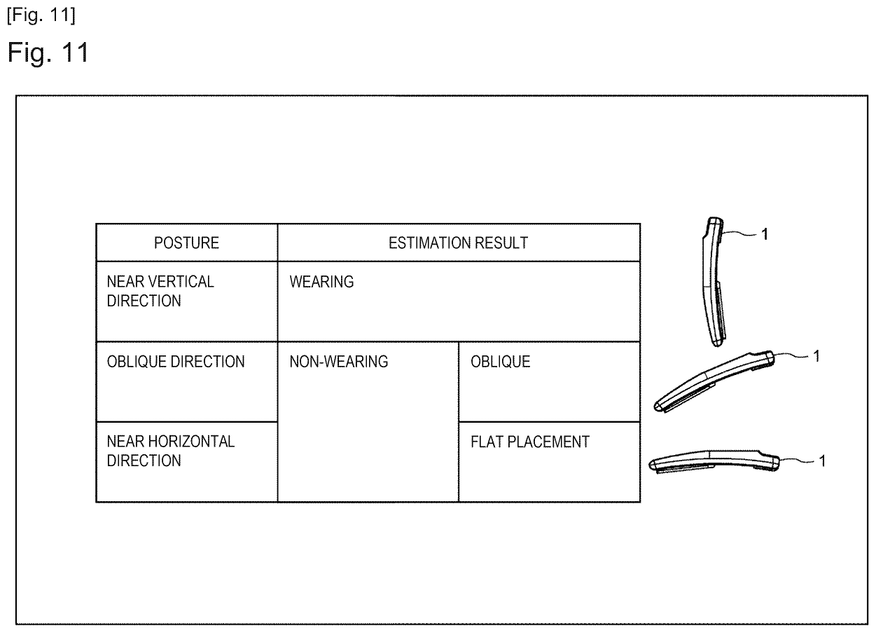

In step S1, the device state estimation unit 62 determines whether a wearing state has lasted for a specified time (for example, three seconds) or more. Specifically, the device state estimation unit 62 estimates a posture of the temperature control device 1 based on the acceleration of the temperature control device 1 detected by the acceleration sensor 21. For example, as illustrated in FIG. 11, the device state estimation unit 62 estimates, based on the posture of the temperature control device 1, whether the temperature control device 1 is worn by the user.

For example, when an angle of the long side direction of the temperature control device 1 with respect to the vertical direction (the gravity direction) is represented as θ (−90 degrees ≤θ≤90 degrees), for example, when −α≤θ≤α (for example, α=10 degrees), that is, when the long side direction of the temperature control device 1 faces the substantially vertical direction, the device state estimation unit 62 estimates that the temperature control device 1 is worn by the user. On the other hand, for example, when θ<−α or θ>α, that is, when the long side direction of the temperature control device 1 tilts from the vertical direction, the device state estimation unit 62 estimates that the temperature control device 1 is not worn by the user.

For example, when −β<θ<−α or α<θ<β (for example, β=80 degrees), the device state estimation unit 62 determines that the temperature control device 1 faces an oblique direction. Further, for example, when −90 degrees ≤θ≤−β or β≤θ≤90 degrees, the device state estimation unit 62 estimates that the temperature control device 1 is placed flat on a table or the like.

Note that, for example, when the user state estimation unit 61 estimates that the user is walking or running, the device state estimation unit 62 corrects an estimation result of the posture of the temperature control device 1. Accordingly, for example, when the user walks or runs while carrying a bag or the like storing the temperature control device 1, it is possible to prevent the temperature control device 1 from being erroneously estimated as being worn by the user.

The device state estimation unit 62 determines whether a wearing state, that is, a state in which the temperature control device 1 is worn by the user has lasted for a specified time or longer. This determination processing is repeatedly executed until it is determined that the wearing state has lasted for the specified time or longer. When it is determined that the wearing state has lasted for the specified time or longer, the processing proceeds to step S2. The device state estimation unit 62 notifies the device state control unit 63 that the wearing state has lasted for the specified time or longer.

In step S2, the device state control unit 63 determines whether the temperature control device 1 is set in the WARM mode. When it is determined that the temperature control device 1 is not set in the WARM mode, that is, the temperature control device 1 is set in the COOL mode or the SMART COOL mode, the processing proceeds to step S3.

In step S3, the device state control unit 63 determines whether the temperature of a low temperature part is equal to or higher than a threshold.

Note that, in the auto start control processing, the environment temperature is used for determination processing for the temperature of the low temperature part. When option setting for using the body surface temperature has been performed, both of the environment temperature and the body surface temperature are used.

When only the environment temperature is used, the device state control unit 63 determines that, when the environment temperature is lower than the threshold, the temperature of the low temperature part is lower than the threshold. The processing proceeds to step S4.

When both of the environment temperature and the body surface temperature are used, the device state control unit 63 determines that, when both of the environment temperature and the body surface temperature are lower than the thresholds, the temperature of the low temperature part is lower than the threshold. The processing proceeds to step 34.

In step S4, the device state control unit 63 determines whether a rising rate of the temperature of the low temperature part is equal to or higher than a threshold.

When only the environment temperature is used, the device state control unit 63 determines that, when a rising rate of the environment temperature is lower than a threshold, the rising rate of the temperature of the low temperature part is lower than the threshold. The processing returns to step S1.

When both of the environment temperature and the body surface temperature are used, the device state control unit 63 determines that, when rising rates of both of the environment temperature and the body surface temperature are lower than thresholds, the rising rate of the temperature of the low temperature part is lower than the threshold. The processing returns to step S1.

Note that change rates (rising rates or falling rates) of the environment temperature and the body surface temperature are calculated by converting values obtained by time-differentiating measurement values of the temperatures into binary values of 0 or 1 using a threshold and further calculating a moving average of the binary values.

Thereafter, processing in step S1 and subsequent steps is executed.

On the other hand, in step S4, when only the environment temperature is used, the device state control unit 63 determines that, when the rising rate of the environment temperature is equal to or higher than the threshold, the rising rate of the temperature of the low temperature part is equal to or higher than the threshold. The processing proceeds to step S5.

When both of the environment temperature and the body surface temperature are used, the device state control unit 63 determines that, when the rising rate of at least one of the environment temperature and the body surface temperature is equal to or higher than the threshold, the rising rate of the temperature of the low temperature part is equal to or higher than the threshold. The processing proceeds to step S5.

This is assumed to be, for example, a case in which the user has moved from a cool environment to a hot environment.

In step S3, when only the environment temperature is used, the device state control unit 63 determines that, when the environment temperature is equal to or higher than the threshold, the temperature of the low temperature part is equal to or higher than the threshold. The processing in step S4 is skipped. The processing proceeds to step S5.

When both of the environment temperature and the body surface temperature are used, the device state control unit 63 determines that, when at least one of the environment temperature and the body surface temperature is equal to or higher than the threshold, the temperature of the low temperature part is equal to or higher than the threshold. The processing in step S4 is skipped. The processing proceeds to step S5.

This is assumed to be, for example, a case in which the user is present in a hot environment.

In step S5, the temperature control device 1 starts the cooling operation. Specifically, the device state control unit 63 turns on the temperature control device 1. The device state control unit 63 instructs the temperature control unit 64 to start the cooling operation. The temperature control unit 64 starts driving of the Peltier element 11 and the fan 13 and starts the cooling operation.

Thereafter, the auto start control processing ends.

On the other hand, when it is determined in step S2 that the temperature control device 1 is set in the WARM mode, the processing proceeds to step S6.

In step S6, the device state control unit 63 determines whether the temperature of the low temperature part is equal to or lower than the threshold.

When only the environment temperature is used, the device state control unit 63 determines that, when the environment temperature is higher than the threshold, the temperature of the low temperature part is higher than the threshold. The processing proceeds to step S7.

When both of the environment temperature and the body surface temperature are used, the device state control unit 63 determines that, when both of the environment temperature and the body surface temperature are higher than the thresholds, the temperature of the low temperature part is higher than the threshold. The processing proceeds to step S7.

Note that the thresholds of the processing in step S3 and the processing in step S6 may be separately set or may be common.

In step S7, the device state control unit 63 determines whether a falling rate of the temperature of the low temperature part is equal to or higher than a threshold.

When only the environment temperature is used, the device state control unit 63 determines that, when a falling rate of the environment temperature is lower than a threshold, the falling rate of the temperature of the low temperature part is lower than the threshold. The processing returns to step S1.

When both of the environment temperature and the body surface temperature are used, the device state control unit 63 determines that, when falling rates of both of the environment temperature and the body surface temperature are lower than thresholds, the falling rate of the temperature of the low temperature part is lower than the threshold. The processing returns to step S1.

Thereafter, the processing in step S1 and subsequent steps is executed.

On the other hand, in step S7, when only the environment temperature is used, the device state control unit 63 determines that, when the falling rate of the environment temperature is equal to or higher than the threshold, the falling rate of the temperature of the low temperature part is equal to or higher than the threshold. The processing proceeds to step S8.

When both of the environment temperature and the body surface temperature are used, the device state control unit 63 determines that, when the falling rate of at least one of the environment temperature and the body surface temperature is equal to or higher than the threshold, the falling rate of the temperature of the low temperature part is equal to or higher than the threshold. The processing proceeds to step S8.

This is assumed to be, for example, a case in which the user has moved from a warm environment to a cold environment.

Note that the thresholds of the processing in step S4 and the processing in step S7 may be separately set or may be common.

In step S6, when only the environment temperature is used, the device state control unit 63 determines that, when the environment temperature is equal to or lower than the threshold, the temperature of the low temperature part is equal to or lower than the threshold. The processing in step S7 is skipped. The processing proceeds to step S8.

When both of the environment temperature and the body surface temperature are used, the device state control unit 63 determines that, when at least one of the environment temperature and the body surface temperature is equal to or lower than the threshold, the temperature of low temperature part is equal to or lower than the threshold. The processing in step S7 is skipped. The processing proceeds to step S8.

This is assumed to be, for example, a case in which the user is present in a cold environment.

In step S8, the temperature control device 1 starts the warming operation. Specifically, the device state control unit 63 turns on the temperature control device 1. The device state control unit 63 instructs the temperature control unit 64 to start the warming operation. The temperature control unit 64 starts driving of the Peltier element 11 and the fan 13 and starts the warming operation.

Thereafter, the auto start control processing ends.

As explained above, the cooling operation or the warming operation is started based on the environment temperature and the change amount of the environment temperature. Accordingly, it is possible to start cooling or warming of the user quickly and appropriately responding to a change in a surrounding environment of the temperature control device 1 (the user).

Further, since the body surface temperature and the change amount of the body surface temperature are used as an option, it is possible to start cooling or warming of the user more quickly and appropriately responding to a change in a surrounding environment of the temperature control device 1 (the user).

### <Auto Stop Control Processing>

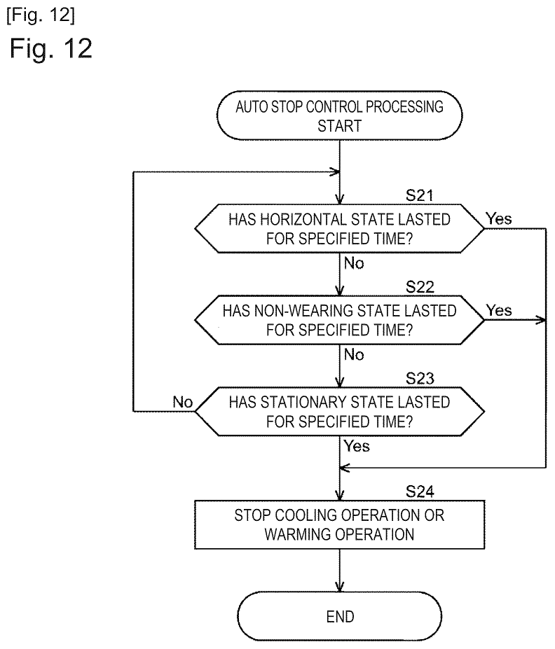

Subsequently, auto stop control processing executed by the temperature control device 1 is explained with reference to a flowchart of FIG. 12. This processing is processing executed at the time of transition from the ON state to the standby state in the state transition diagram of FIG. 8.

In step S21, the device state estimation unit 62 determines whether a horizontal state has lasted for a specified time or longer. Specifically, as in the processing in step S1 in FIG. 10, the device state estimation unit 62 estimates a posture and a wearing state of the temperature control device 1. The device state estimation unit 62 determines, based on an estimation result of the posture of the temperature control device 1, whether the horizontal state has lasted for the specified time or longer. When it is determined that the horizontal state has not lasted for the specified time or longer, the processing proceeds to step S22.

In step S22, the device state estimation unit 62 determines, based on an estimation result of the wearing state of the temperature control device 1, whether a non-wearing state has lasted for the specified time. When it is determined that the non-wearing state has not lasted for the specified time, the processing proceeds to step S23.

In step S23, the device state estimation unit 62 determines, based on the estimation result of the posture of the temperature control device 1, whether a stationary state has lasted for a specified time. The stationary state is, for example, a state in which a change in the posture of the temperature control device 1 is within a predetermined range. When it is determined that the stationary state has not lasted for the specified time, the processing returns to step S21.

Note that the specified times of the processing in step S21 to step S23 may be separately set or may be common.

Thereafter, the processing in step S21 to step S23 is repeatedly executed until it is determined in step S21 that the horizontal state has lasted for the specified time, it is determined in step S22 that the non-wearing state has lasted for the specified time, or it is determined in step S23 that the stationary state has lasted for the specified time.

On the other hand, when it is determined in step S21 that the horizontal state has lasted for the specified time, the processing proceeds to step S24.

Accordingly, for example, a state in which the temperature control device 1 is removed from the user and placed flat on a desk or the like is detected.

When it is determined in step S22 that the non-wearing state has lasted for the specified time, the processing proceeds to step S24.

Further, when it is determined in step S23 that the stationary state has lasted for the specified time, the processing proceeds to step S24.

For example, a state in which the temperature control device 1 is removed from the user and placed in a state other than the flat placement or stored in a bag or the like moving together with the user is detected by the determination processing in step S22 and step S23.

In step S24, the temperature control device 1 stops the cooling operation or the warming operation. Specifically, the horizontal state, the non-wearing state, or the stationary state has lasted from the specified time, whereby the device state estimation unit 62 estimates that the temperature control device 1 has been removed from the user. The device state estimation unit 62 notifies the device state control unit 63 that the temperature control device 1 has been removed from the user.

The device state control unit 63 instructs the temperature control unit 64 to stop the cooling operation or the warming operation. In response, the temperature control unit 64 stops the Peltier element 11 and the fan 13. The device state control unit 63 sets the state of the temperature control device 1 to the standby state and turns off the temperature control device 1.

Thereafter, the auto stop control processing ends.

As explained above, when the temperature control device 1 is removed from the user, the temperature control device 1 is automatically turned off. Accordingly, power saving of the temperature control device 1 is realized and a use time of the battery 14 can be extended.

### <Temperature Control Processing>

Subsequently, temperature control processing of the temperature control device 1 is explained.

For example, when the operation mode is set to the COOL mode or the WARM mode, the temperature control device 1 (the temperature control unit 64) controls output of the Peltier element 11 based on temperature setting. In this case, the output of the Peltier element 11 is fixed based on the set temperature and temperature control corresponding to a surrounding environment or the like is not performed.

On the other hand, when the temperature control device 1 is set in the SMART COOL mode, the temperature control device 1 automatically adjusts a target temperature and controls on/off of the cooling operation according to a surrounding environment or the like.

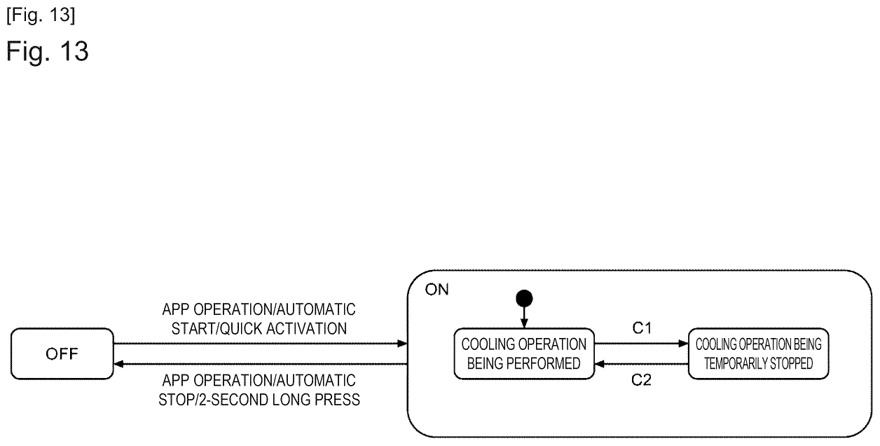

Here, temperature control processing of the temperature control device 1 in the case in which the temperature control device 1 is set in the SMART COOL mode is explained with reference to FIG. 13 to FIG. 17.

FIG. 13 illustrates a state transition diagram of the temperature control device 1 in the SMART COOL mode.

The temperature control device 1 transitions between two kinds of states, that is, an OFF state in which the SMART COOL mode is turned off and an ON state in which the SMART COOL mode is turned on.

The temperature control device 1 transitions from the OFF state to the ON state according to, for example, App operation, automatic start, or quick activation. the APP Operation is operation for turning on the SMART COOL mode using an APP on the wearable device 2. The automatic start is, for example, a function of automatically turning on the SMART COOL mode when the temperature control device 1 is turned on. The quick activation is, for example, operation for quickly turning on the SMART COOL mode by performing predetermined operation on the interface unit 51.

The temperature control device 1 transitions from the ON state to the OFF state according to, for example, APP operation, automatic stop, or two-second long press. The APP operation is operation for turning off the SMART COOL mode using the APP on the wearable device 2. The automatic stop is, for example, a function of automatically turning off the SMART COOL mode when the temperature control device 1 is turned off. The two-second long press is, for example, operation for turning off the SMART COOL mode by pressing a predetermined button of the interface unit 51 long for two or more seconds.

In the case of the ON state, the temperature control device 1 transitions between states of cooling operation being performed and cooling operation being temporarily stopped. The cooling operation being performed is a state in which the temperature control device 1 is executing the cooling operation. The cooling operation being temporarily stopped is a state in which the temperature control device 1 temporarily stops the cooling operation.

For example, the temperature control device 1 transitions to the cooling operation being temporarily stopped when a transition condition C1 is satisfied in the cooling operation being performed. The transition condition C1 is that, for example, a state in which an environment temperature is equal to or lower than T1° C. and a heat absorbing surface temperature is equal to or lower than (set temperature +T2)°C. has lasted for a predetermined time. T1 is set, for example, in a range of 28 to 32° C. T2 is set, for example, in a range of 1 to 2° C.

For example, when a transition condition C2 is satisfied in the cooling operation being temporarily stopped, the temperature control device 1 transitions to the cooling operation being performed. The transition condition C2 is that, for example, the environment temperature is T3° C. or higher or the heat absorbing surface temperature is (the set temperature +T4)° C. or higher. T3 is set, for example, in a range of 30 to 34° C. T4 is set, for example, in a range of 2 to 4° C.

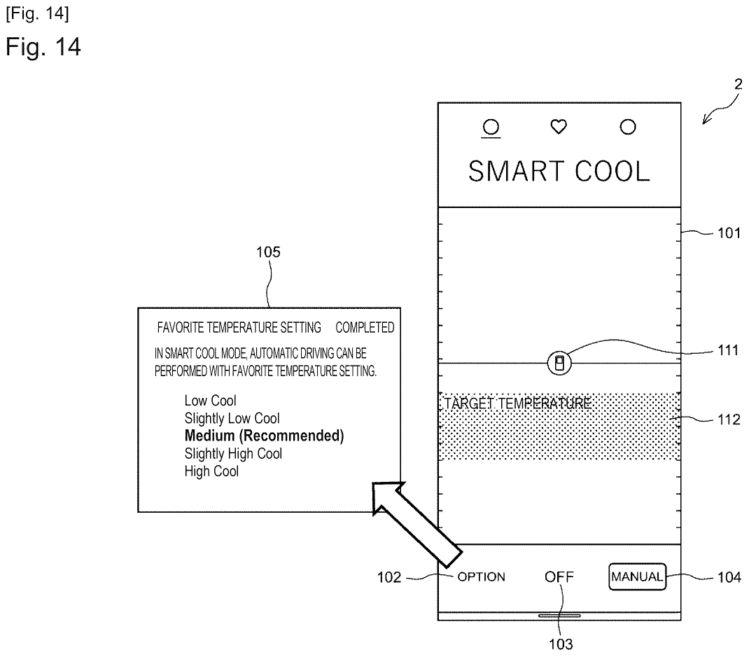

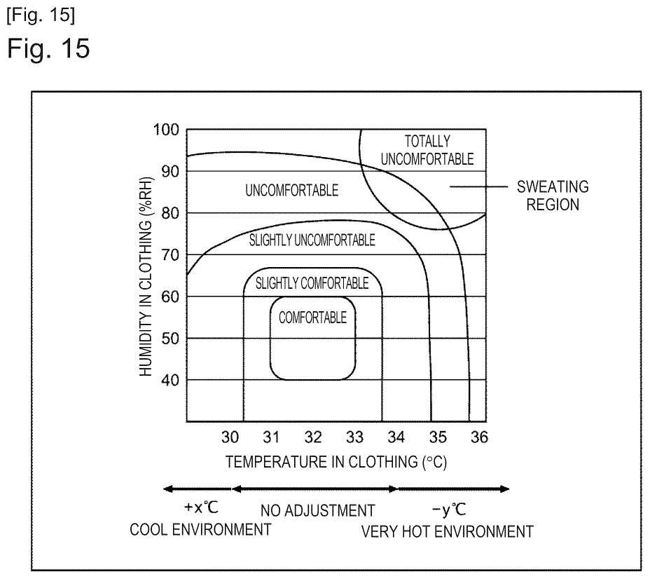

Subsequently, a method of controlling a target temperature in the SMART COOL mode is explained with reference to FIG. 14 and FIG. 15.

When the temperature control device 1 is set in the SMART COOL mode, the temperature control device 1 adjusts the target temperature with reference to a set temperature based on an environment temperature and a behavior of the user.

Note that, since the environment temperature and the behavior of the user are different factors that affect a feeling temperature of the user, the environment temperature and the behavior of the user are respectively independently detected or estimated and separately used for adjusting the target temperature.

FIG. 14 illustrates an example of a setting screen for the SMART COOL mode displayed on the wearable device 2.

The setting screen includes a window 101, an option button 102, an OFF button 103, and a manual button 104.

An icon 111 indicating a current temperature and a target temperature region 112 indicating the target temperature are displayed on the window 101.

The position of the icon 111 moves in the up-down direction according to, for example, a change in the heat absorbing surface temperature.

The position of the target temperature region 112 moves in the up-down direction according to a change in the target temperature.

When the option button 102 is pressed, a favorite temperature setting window 105 is displayed. On the favorite temperature setting window 105, the user can select a desired set temperature out of five stages of “Low Cool”, “Slightly Low Cool”, “Medium (Recommended)”, “Slightly High Cool”, and “High Cool”.

When a favorite temperature is set to “Low Cool”, for example, the set temperature is set to 29 to 31° C. When the favorite temperature is set to “Slightly Low Cool”, for example, the set temperature is set to 28 to 30° C. When the favorite temperature is set to “Medium (Recommended)”, for example, the set temperature is set to 27 to 29°C. When the favorite temperature is set to “Slightly High Cool”, for example, the set temperature is set to 26 to 28° C. When the favorite temperature is set to “High Cool”, for example, the set temperature is set to 25 to 27° C. The temperature control unit 64 sets an initial value of the target temperature to the set temperature set by the user.

When the OFF button 103 is pressed, the temperature control device 1 is turned off.

When the manual button 104 is pressed, for example, a screen for setting the operation mode to the COOL mode or the WARM mode and setting the set temperature is displayed.

FIG. 15 illustrates an example of an indicator representing the comfort in clothing. The horizontal axis of FIG. 15 indicates the temperature (a unit is °C.) in the clothing and the vertical axis indicates a relative humidity (a unit is % RH) in the clothing.

As illustrated in FIG. 15, comfort and discomfort of a person change according to the temperature and the humidity in the clothing.

On this matter, the temperature control device 1 adjusts the target temperature according to the temperature inside the clothing of the user (an environment temperature).

For example, when the environment temperature is within a predetermined range (for example, 30 to 34° C.), the temperature control unit 64 does not change the target temperature from a current value.

On the other hand, for example, when the environment temperature is lower than the predetermined range explained above, that is, in the case of a cool environment, the temperature control unit 64 raises the target temperature by +x° C. (for example, +1 to +2° C.) from the current value.

For example, when the environment temperature is higher than the predetermined range explained above, that is, in the case of a very hot environment, the temperature control unit 64 lowers the target temperature by −y°C. (for example, −0.5 to −1° C.) from the current value.

Note that a width of raising the target temperature (+1 to +2° C.) is larger than a width of lowering the target temperature (−0.5 to −1° C.). However, for example, both the widths may be set to the same value. The width of raising the target temperature can also be set smaller than the width of lowering the target temperature.

Further, the temperature control unit 64 adjusts the target temperature based on a behavior of the user.

For example, when a state in which the user moves the body by walking or running (hereinafter referred to as active state) has lasted, the temperature control unit 64 lowers the target temperature for a while after an end of the active state. Specifically, for example, after a state in which the user state estimation unit 61 estimates that the user is in the active state has lasted for a specified time (for example, five minutes), when estimating that the active state has stopped, the temperature control unit 64 lowers the target temperature by a predetermined temperature (for example, −0.5 to −1° C.) for a predetermined time (for example, thirty seconds).

For example, a person more often feels heat after an activity than during the activity. For that reason, the target temperature is lowered for the predetermined time after the activity in this way.

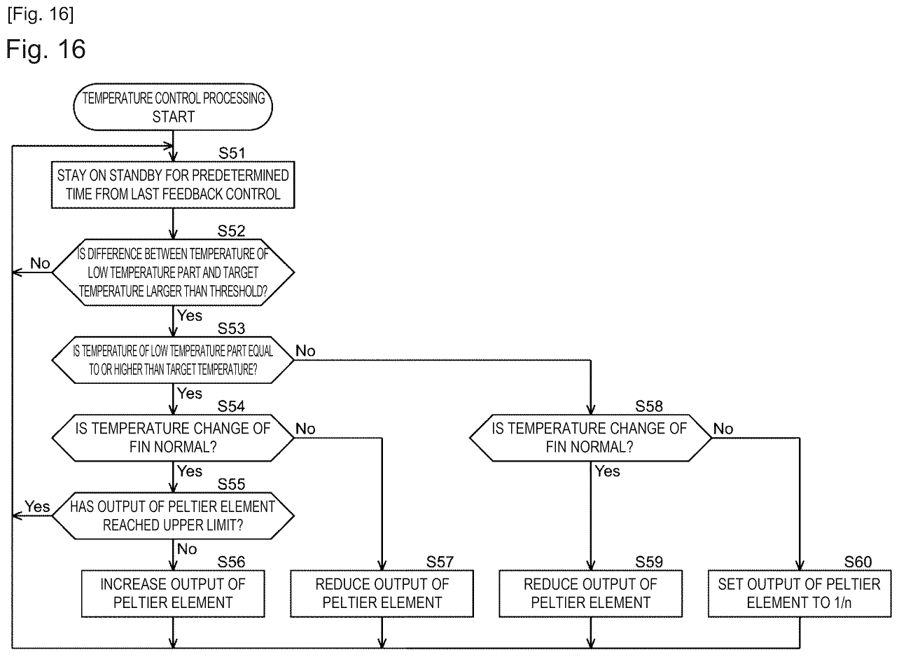

Subsequently, an example of temperature control processing of the temperature control device 1 in the case in which a state of the temperature control device 1 is the cooling operation being performed illustrated in FIG. 13 is explained with reference to a flowchart of FIG. 16.

In step S51, the temperature control unit 64 stays on standby for a predetermined time (for example, five to ten seconds) from the last feedback control.

In step S52, the temperature control unit 64 determines whether the difference between the temperature of the low temperature part and the target temperature is larger than a threshold. For example, when the difference between the heat absorbing surface temperature and the target temperature is not larger than the threshold, the temperature control unit 64 determines that the difference between the temperature of the low temperature part and the target temperature is not larger than the threshold. The processing returns to step S51. The threshold is set, for example, in a range of ±0.1 to ±1.0° C.

Note that, as explained above, the target temperature does not always coincide with the set temperature and is adjusted according to a situation.

Thereafter, the processing in step S51 and step S52 is repeatedly executed until it is determined in step S52 that the difference between the temperature of the low temperature part and the target temperature is larger than the threshold.

On the other hand, in step S52, for example, when the difference between the heat absorbing surface temperature and the target temperature is larger than the threshold, the temperature control unit 64 determines that the difference between the temperature of the low temperature part and the target temperature is larger than the threshold. The processing proceeds to step S53.

In step S53, the temperature control unit 64 determines whether the temperature of the low temperature part is equal to or higher than the target temperature. For example, when the heat absorbing surface temperature is equal to or higher than the target temperature, the temperature control unit 64 determines that the temperature of the low temperature part is equal to or higher than the target temperature. The processing proceeds to step S54.

In step S54, the temperature control unit 64 determines whether a temperature change of the fin 12 is normal. For example, when the fin temperature is lower than a predetermined threshold, the temperature control unit 64 determines that the temperature change of the fin 12 is normal. The processing proceeds to step S55.

In step S55, the temperature control unit 64 determines whether output of the Peltier element 11 has reached an upper limit. When it is determined that the output of the Peltier element 11 has not reached the upper limit, the processing proceeds to step S56.

In step S56, the temperature control unit 64 increases the output of the Peltier element 11. For example, the temperature control unit 64 increases the output of the Peltier element 11 by a predetermined rate (for example, 1 to 50%). Accordingly, the temperature on the heat absorption side of the Peltier element 11 falls (the cooling temperature falls).

Thereafter, the processing returns to step S51. The processing in step S51 and subsequent steps is executed.

On the other hand, when it is determined in step S55 that the output of the Peltier element 11 has reached the upper limit, the processing returns to step S51. The processing in step S51 and subsequent steps is executed. In this case, the output of the Peltier element 11 is not changed.

In step S54, for example, when the fin temperature is equal to or higher than the predetermined threshold, the temperature control unit 64 determines that the temperature change of the fin 12 is abnormal. The processing proceeds to step S57.

This is assumed to be a case in which the temperature difference between the heat absorption side and the heat dissipation side is excessively large because of an abnormality of the Peltier element 11 or the fin 12 is not normally cooled because of a factor such as an abnormality of the fan 13 or closing of the intake ports 101 or the exhaust ports 10E.

In step S57, the temperature control unit 64 reduces the output of the Peltier element 11. For example, the temperature control unit 64 reduces the output of the Peltier element 11 by a predetermined rate (for example, 1 to 50%). Accordingly, the temperature on the heat absorption side of the Peltier element 11 rises (the cooling temperature rises).

Thereafter, the processing returns to step S51. The processing in step S51 and subsequent steps is executed.

On the other hand, in step S53, for example, when the heat absorbing surface temperature is lower than the target temperature, the temperature control unit 64 determines that the temperature of the low temperature part is lower than the target temperature. The processing proceeds to step S58.

In step S58, as in the processing in step S54, it is determined whether the temperature change of the fin 12 is normal. When it is determined that the temperature change of the fin 12 is normal, the processing proceeds to step S59.

In step S59, the temperature control unit 64 reduces the output of the Peltier element 11. For example, the temperature control unit 64 reduces the output of the Peltier element 11 by a predetermined rate (for example, 1 to 50%). Accordingly, the temperature on the heat absorption side of the Peltier element 11 rises (the cooling temperature rises).

Thereafter, the processing returns to step S51. The processing in step S51 and subsequent steps is executed.

On the other hand, when it is determined in step S58 that the temperature change of the fin 12 is abnormal, the processing proceeds to step S60.

In step S60, the temperature control unit 64 sets the output of the Peltier element 11 to 1/n (for example, 1/10 to ½). Accordingly, the temperature on the heat absorption side of the Peltier element 11 rises (the cooling temperature rises).

Thereafter, the processing returns to step S51. The processing in step S51 and subsequent steps is executed.

As explained above, the output of the Peltier element 11 is controlled and the temperature of the Peltier element 11 (the element temperature) is controlled such that the temperature of the low temperature part (for example, the heat absorbing surface temperature) approaches the target temperature. As explained above, the target temperature is adjusted based on the surrounding environment (for example, the environment temperature) and the state of the user (for example, the behavior of the user). Therefore, the temperature of the Peltier element 11 is controlled and the cooling temperature of the temperature control device 1 is appropriately adjusted based on the surrounding environment and the state of the user. As a result, the comfort of the user is improved.

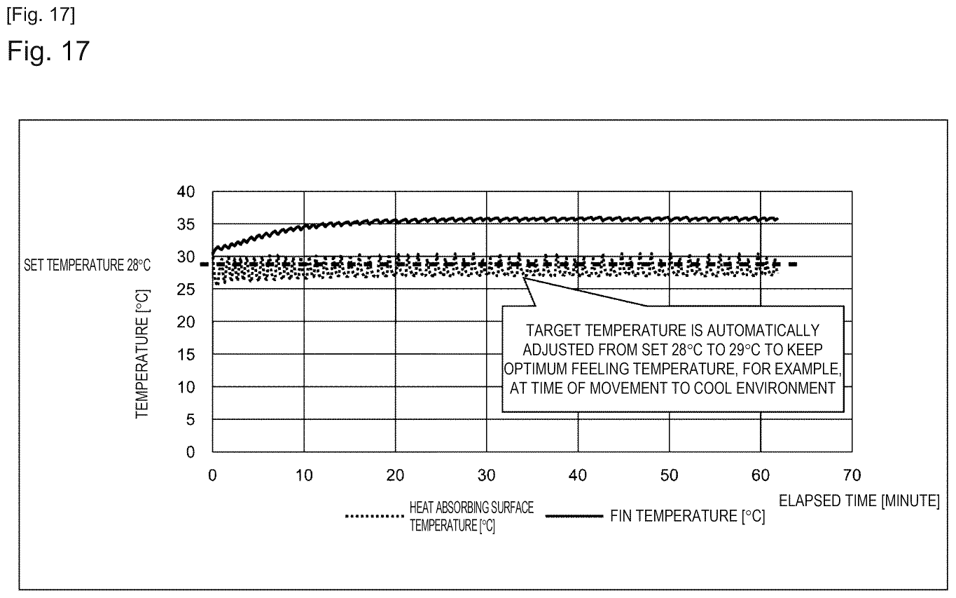

For example, FIG. 17 illustrates an example of time-series changes of the heat absorbing surface temperature and the fin temperature. The horizontal axis indicates an elapsed time (a unit is minute) from when the temperature control device 1 is worn and the vertical axis indicates temperature (a unit is ° C.).

In this example, the set temperature is set to 28° C. For example, when the user has moved to a cool environment during wearing the temperature control device 1, the target temperature is automatically adjusted from 28° C. to 29° C., whereby the heat absorbing surface temperature is kept substantially constant. Accordingly, a feeling temperature optimum for the user is kept and the comfort is improved.

### 2. Modifications

### <Modification Concerning the Temperature Control

In the above explanation, an example is explained in which the environment sensor 19 measures the temperature inside the clothing as the environment temperature. However, for example, the environment sensor 19 may measure, instead of the temperature inside the clothing, the outside air temperature outside the clothing as the environment temperature or measure both of the temperature inside the clothing and the outside air temperature. Since the outside air temperature can be measured, the temperature control device 1 is capable of more appropriately executing temperature adjustment and the comfort of the user is improved.

For example, in the above explanation, an example is explained in which the temperature control device 1 automatically adjusts the cooling temperature. However, the temperature control device 1 may be able to automatically adjust a warming temperature. In this case, for example, by using the outside air temperature measured by the environment sensor 19, the temperature control unit 64 is capable of more appropriately adjusting the warming temperature.

For example, the temperature control device 1 may be able to automatically switch the cooling operation and the warming operation. In this case, for example, by using the outside air temperature measured by the environment sensor 19, the device state control unit 63 is capable of more appropriately switching the cooling operation and the warming operation. Note that, for example, the device state control unit 63 may switch the cooling operation and the warming operation considering a season and the like in addition to the outside air temperature.

### <Modification Concerning the Method of Adjusting the Target Temperature>

For example, the target temperature may be adjusted based on biological information of the user detected by a biological sensor or the like. As such biological information, for example, an activity amount (for example, a consumed calorie) and a heart rate of the user are assumed.

For example, the target temperature may be adjusted based on both of the behavior and the biological information of the user.

### <Modification Concerning the Method of Detecting Presence or Absence of Wearing the Temperature Control Device 1>

In the above explanation, an example is explained in which the presence or absence of wearing (wearing and removing) the temperature control device 1 is detected using the acceleration sensor 21. However, other methods can also be adopted.

For example, the presence or absence of wearing the temperature control device 1 may be detected by using other sensors such as the body surface sensor 20, an angular velocity sensor, and a touch sensor other than the acceleration sensor 21 or combining the acceleration sensor 21 and the other sensors.

### <Modification Concerning the Method of Wearing the Temperature Control Device 1>

In the above explanation, an example is explained in which the temperature control device 1 is stored in the pocket 81P of the underwear 81 and the temperature control device 1 is worn to be disposed near the cervical spine of the user. However, a method of wearing the temperature control device 1 and a wearing position of the temperature control device 1 is not particularly limited.

For example, the temperature control device 1 may be stored in a pocket provided in an underwear or clothing other than the underwear 81. The temperature control device 1 may be worn by the user using, for example, a strap or a supporter.

For example, the user can wear the temperature control device 1 to any part of the body that the user desires to cool or warm other than the cervical spine or hold and place the temperature control device 1 on the part of the body. The user can wear the temperature control device 1 to directly touch the skin or can wear the temperature control device 1 to indirectly touch the skin via cloth or the like.

### Other Modifications

The present technology is explained above with reference to the embodiment and the modifications. However, the present technology is not limited to the embodiment and the like explained above and can be variously modified. For example, the components, the disposition, and the like of the temperature control device 1 exemplified in the embodiment and the like explained above are only an example. The temperature control device 1 does not need to include all of the components and may further include other components.

In the embodiment and the like explained above, the Peltier element 11 is explained as a specific example of the temperature changer of the present disclosure. However, the temperature changer may be configured by another element.

In the embodiment and the like, an example in which the person wears the temperature control device 1 is explained as an example of use of the temperature control device 1. However, the temperature control device 1 may cool or warm a thing such as a beverage. Alternatively, a smartphone and the like can also be cooled by the temperature control device 1.

### 3. Others

The series of processing explained above can be executed by hardware or can be executed by software. When the series of processing is executed by the software, a program configuring the software is installed in a computer. Here, the computer includes a computer incorporated in dedicated hardware and, for example, a general-purpose personal computer capable of executing various functions by installing various programs.

Note that the program to be executed by the computer may be a program in which processing is performed in time series according to the order explained in the present specification or may be a program in which processing is performed in parallel or at necessary timing such as timing when the program is invoked.

In the present specification, a system means a set of a plurality of components (devices, modules (components), and the like) and it does not matter whether all the components are present in the same housing. Therefore, both of a plurality of devices housed in separate housings and connected via a network and one device in which a plurality of modules are housed in one housing are the system.

Further, the embodiment of the present technology is not limited to the embodiment explained above and can be variously changed without departing from the gist of the present technology.

For example, the present technology can adopt a configuration of cloud computing in which one function is shared and processed in cooperation by a plurality of devices via a network.

The steps explained in the flowcharts above can be shared and executed by a plurality of devices besides being executed by one device.

Further, when a plurality of kinds of processing are included in one step, the plurality of kinds of processing included in the one step can be shared and executed by a plurality of devices besides being executed by one device.

### <Combination Example of Configurations>

The present technology can also take configurations explained below.

(1) A temperature control device including:
an environment sensor that measures an environment temperature that is temperature of a surrounding environment;
a temperature changer used for cooling at least a body surface of a user; and
a temperature control unit that controls, based on the environment temperature, an element temperature that is temperature of the temperature changer.

(2) The temperature control device described in (1) above, wherein the temperature control unit adjusts a target temperature based on the environment temperature and controls the element temperature based on the target temperature.

(3) The temperature control device described in (2) above, further including a heat absorbing surface sensor that measures a heat absorbing surface temperature that is temperature of a heat absorbing surface of the temperature changer, wherein
when a difference between the heat absorbing surface temperature and the target temperature is larger than a predetermined range, the temperature control unit lowers the element temperature when the heat absorbing surface temperature is higher than the target temperature and raises the element temperature when the heat absorbing surface temperature is lower than the target temperature.

(4) The temperature control device described in (3) above, including:
a heat dissipation member used for heat dissipation of the temperature changer; and
a heat dissipation member sensor that measures a heat dissipation member temperature that is temperature of the heat dissipation member, wherein
the temperature control unit raises the element temperature when the heat dissipation member temperature is equal to or higher than a predetermined threshold.

(5) The temperature control device described in any one of (2) to (4) above, wherein the temperature control unit sets an initial value of the target temperature based on a user setting.

(6) The temperature control device described in any one of (1) to (5) above, wherein the temperature control unit raises the element temperature when the environment temperature is lower than a predetermined range and lowers the element temperature when the environment temperature is higher than the predetermined range.

(7) The temperature control device described in (6) above, wherein a width of raising the element temperature is larger than a width of lowering the element temperature.

(8) The temperature control device described in any one of (1) to (7) above, including:
a device state estimation unit that estimates presence or absence of wearing by the user; and
a device state control unit that controls, based on the presence or absence of wearing by the user, at least one of a start and a stop of a cooling operation by the temperature changer.

(9) The temperature control device described in (8) above, wherein the device state control unit controls the start of the cooling operation further based on at least one of the environment temperature and a change rate of the environment temperature.

(10) The temperature control device described in (9) above, further including a body surface sensor that measures a body surface temperature that is temperature of the body surface, wherein
the device state control unit controls the start of the cooling operation further based on at least one of the body surface temperature and a change rate of the body surface temperature.

(11) The temperature control device described in any one of (8) to (10) above, wherein
the device state estimation unit further estimates a posture of the temperature control device, and
the device state control unit controls the stop of the cooling operation further based on the posture of the temperature control device.

(12) The temperature control device described in (11) above, wherein the device state estimation unit estimates the presence or absence of wearing by the user based on the posture of the temperature control device.

(13) The temperature control device described in any one of (1) to (12) above, further including a user state estimation unit that estimates a state of the user, wherein
the temperature control unit adjusts the element temperature further based on the state of the user.

(14) The temperature control device described in (13) above, wherein
the user state estimation unit estimates at least one of a behavior and an activity amount of the user, and
the temperature control unit adjusts the element temperature based on at least one of the behavior and the activity amount of the user.

(15) The temperature control device described in (14) above, wherein, when a predetermined behavior of the user has lasted for a predetermined time or longer, the temperature control unit lowers the element temperature after the predetermined behavior has stopped.

(16) The temperature control device described in (15) above, wherein the temperature control unit lowers the element temperature for a predetermined time after the predetermined behavior has stopped.

(17) The temperature control device described in any one of (1) to (16) above, wherein the temperature changer is further used for warming the body surface.

(18) The temperature control device described in any one of (1) to (17) above, further including:
a heat dissipation member used for heat dissipation of the temperature changer; and
a fan that performs ventilation of surroundings of the heat dissipation member.

(19) The temperature control device described in any one of (1) to (18) above, wherein the environment temperature is temperature inside clothing of the user.

(20) A temperature control method including a temperature control device:
measuring an environment temperature that is temperature of a surrounding environment; and
controlling, based on the environment temperature, an element temperature that is temperature of a temperature changer used for cooling at least a body surface of a user.

Note that the effects described in the present specification are merely exemplifications and are not limiting. There may be other effects.

### REFERENCE SIGNS LIST

1 Temperature control device, 2 Wearable device,
10 Housing, 11 Peltier element, 12 Fin, 13 Fan, 14 Battery, 15 Circuit board, 16 Heat absorbing surface sensor, 18 Fin sensor, 19 Environment sensor, 20 Body surface sensor, 21 Acceleration sensor, 22 Wireless communication unit, 51 Interface unit, 52 Control unit, 61 User state estimation unit, 62 Device state estimation unit, 63 Device state control unit, 64 Temperature control unit

1. A temperature control device comprising:
an environment sensor that measures an environment temperature that is temperature of a surrounding environment;
a temperature changer used for cooling at least a body surface of a user; and
a temperature control unit that controls, based on the environment temperature, an element temperature that is temperature of the temperature changer.

2. The temperature control device according to claim 1, wherein the temperature control unit adjusts a target temperature based on the environment temperature and controls the element temperature based on the target temperature.

3. The temperature control device according to claim 2, further comprising a heat absorbing surface sensor that measures a heat absorbing surface temperature that is temperature of a heat absorbing surface of the temperature changer, wherein
when a difference between the heat absorbing surface temperature and the target temperature is larger than a predetermined range, the temperature control unit lowers the element temperature when the heat absorbing surface temperature is higher than the target temperature and raises the element temperature when the heat absorbing surface temperature is lower than the target temperature.

4. The temperature control device according to claim 3, comprising:
a heat dissipation member used for heat dissipation of the temperature changer; and
a heat dissipation member sensor that measures a heat dissipation member temperature that is temperature of the heat dissipation member, wherein
the temperature control unit raises the element temperature when the heat dissipation member temperature is equal to or higher than a predetermined threshold.

5. The temperature control device according to claim 2, wherein the temperature control unit sets an initial value of the target temperature based on a user setting.

6. The temperature control device according to claim 1, wherein the temperature control unit raises the element temperature when the environment temperature is lower than a predetermined range and lowers the element temperature when the environment temperature is higher than the predetermined range.

7. The temperature control device according to claim 6, wherein a width of raising the element temperature is larger than a width of lowering the element temperature.

8. The temperature control device according to claim 1, comprising:
a device state estimation unit that estimates presence or absence of wearing by the user; and
a device state control unit that controls, based on the presence or absence of wearing by the user, at least one of a start and a stop of a cooling operation by the temperature changer.

9. The temperature control device according to claim 8, wherein the device state control unit controls the start of the cooling operation further based on at least one of the environment temperature and a change rate of the environment temperature.

10. The temperature control device according to claim 9, further comprising a body surface sensor that measures a body surface temperature that is temperature of the body surface, wherein
the device state control unit controls the start of the cooling operation further based on at least one of the body surface temperature and a change rate of the body surface temperature.

11. The temperature control device according to claim 8, wherein
the device state estimation unit further estimates a posture of the temperature control device, and
the device state control unit controls the stop of the cooling operation further based on the posture of the temperature control device.

12. The temperature control device according to claim 11, wherein the device state estimation unit estimates the presence or absence of wearing by the user based on the posture of the temperature control device.

13. The temperature control device according to claim 1, further comprising a user state estimation unit that estimates a state of the user, wherein
the temperature control unit adjusts the element temperature further based on the state of the user.

14. The temperature control device according to claim 13, wherein
the user state estimation unit estimates at least one of a behavior and an activity amount of the user, and
the temperature control unit adjusts the element temperature based on at least one of the behavior and the activity amount of the user.

15. The temperature control device according to claim 14, wherein, when a predetermined behavior of the user has lasted for a predetermined time or longer, the temperature control unit lowers the element temperature after the predetermined behavior has stopped.

16. The temperature control device according to claim 15, wherein the temperature control unit lowers the element temperature for a predetermined time after the predetermined behavior has stopped.

17. The temperature control device according to claim 1, wherein the temperature changer is further used for warming the body surface.

18. The temperature control device according to claim 1, further comprising:
a heat dissipation member used for heat dissipation of the temperature changer; and
a fan that performs ventilation of surroundings of the heat dissipation member.

19. The temperature control device according to claim 1, wherein the environment temperature is temperature inside clothing of the user.

20. A temperature control method comprising a temperature control device:
measuring an environment temperature that is temperature of a surrounding environment; and
controlling, based on the environment temperature, an element temperature that is temperature of a temperature changer used for cooling at least a body surface of a user.
